(19) United States
(12) Patent Application Publication
(10) Pub. No.: US 2019/0099341 A1
(43) Pub. Date: Apr. 4, 2019

(54) SKIN AND HAIR TREATMENTS

(71) Applicants: Robert B. Aylor, Blue Ash, OH (US); L. Heather Makover, Ft. Myers, FL (US)

(72) Inventors: Robert B. Aylor, Blue Ash, OH (US); L. Heather Makover, Ft. Myers, FL (US)

(21) Appl. No.: 15/725,204

(22) Filed: Oct. 4, 2017

(51) Int. Cl. A61K 8/34; A61K 8/31; A61Q 19/08

(52) U.S. Cl. CPC A61K 8/342; A61K 2800/87; A61Q 19/08; A61K 8/31

(57) ABSTRACT

Linalool and other materials which are skin penetrating vasodilators or which can be made to have such properties are applied, either by themselves or in skin treatment compositions, to the skin to minimize wrinkles and/or improve the skin's appearance. Products containing at least about 1% linalool are packaged in a container with instructions to shake well before use to maintain their suitability tor providing the desired skin benefits during the products' use.

### CROSS-REFERENCE TO RELATED APPLICATIONS

This application is a divisional of U.S. application Ser. No. 13/507,607, filed Jul. 14, 2012 which claims priority from U.S. Provisional Application Ser. No 61/572,443, filed Jul. 15, 2011. all of which are herein incorporated by reference in their entireties.

### BACKGROUND OF THE INVENTION

### Field of the Invention

The invention relates to the improvements in minimizing the occurrence and severity of pain, including muscle, tendon, ligament, etc. pains and especially migraine headaches, either alternately or additionally, providing appearance benefits such as wrinkle reduction and hair maintenance; and to methods, compositions, etc. for providing such improvements.

### Description of Related Art

U.S. Pat. No. 4,371,461, Jones, et al., issued Feb. 1, 1983, claims: A liquid detergent composition containing by weight: (a) from about 10% to about 50% of a detergent surfactant, (b) from 0% to about 15% of a suds stabilizing nonionic surfactant selected from the group consisting of amine oxides, fatty acid amides, and the ethylene oxide condensates of alcohols and alkyl phenols; (c) from about 0.01% to about 0.6% of linalool; and (d) balance water and alcohol said composition being essentially free of aldehydes and primary alcohols with structures like linalool and having a pH in use of less than about 8.5.

The patent does not mention treating migraine headaches and teaches the composition primarily for cleaning dishes while providing a good feel for the hands

US Pat. Appl. 20110092493, Levi, Clark et al., filed Apr. 21, 2011 teaches formulations for transdermal delivery of promethazine which can be used for pain, e.g., from migraine headaches. There is an incidental disclosure of linalool as an optional ingredient.

US Pat. Appl. 20110144191, McLellan, Alexander, et al., filed Jun. 16, 2011, teaches compositions comprising terpene compounds for the treatment of negative sensory phenomena. The compositions mention linalool as an optional ingredient and mention migraine headaches.

There are many internet sites that document in great detail studies that concern linalool, its safety and uses, including:

1-17. (canceled)

18. A skin conditioning product comprising an effective amount of more than about 1% of a mild vasodilator compound selected from the group consisting of limonene, linalool, tetrahydrolinalool, 4-terpineol, Aprol-100 (3,6-dimethyl octane-3-ol), alpha-terpineol, ocimenol, nerolidol, or combinations thereof that can penetrate the skin and that is suitable, with constant use, for providing the benefits of minimizing wrinkles and improving the appearance and health of skin, said mild vasodilator compound being physically unstable in said skin conditioning product and said product being in a container in association with instructions to apply the dispensed product to skin such as wrinkled or rough skin to minimize the wrinkles and improve the appearance and health of the skin and also with instructions to shake said product well to ensure that the product that is dispensed each time during the use of said product contains an effective amount of said mild vasodilator compound to provide the said benefits.

19. The product of claim 18 wherein said mild vasodilator compound is linalool at a level of at least about 1% by volume

20. The product of claim 19 wherein said linalool is at a level of at least about 2%.

21. The product of claim 19 wherein said linalool is at a level of at least about 3%.

22. The product of claim 18 wherein said mild vasodilator compound is at a level of at least about 1% and the product is aqueous.

23. The product of claim 22 wherein said mild vasodilator compound is at a level of at least about 2%.

24. The product of claim 23 wherein said mild vasodilator compound is at a level of at least about 3%.

25. The product of claim 18 wherein said mild vasodilator compound is at a level of more than about 2% in the product.

26. A method for treating skin comprising shaking the product of claim 18 well and then applying an effective amount of said product to skin that is wrinkled.

27. The method of claim 26 wherein the said mild vasodilator compound is linalool and the level is more than about 2%.

28. The method of claim 27 wherein the amount of said linalool in said skin conditioning product is at least about 3%.

29. The method of claim 26 wherein the amount of said mild vasodilator compound in said skin conditioning product is at least about 2%

30. The method of claim 26 wherein the amount of said mild vasodilator compound in said skin conditioning product is at least about 3%.
